(19) United States
(12) Patent Application Publication
(10) Pub. No.: US 2026/0364363 A1
(43) Pub. Date: Nov. 19, 2026

(54) SYSTEMS AND METHODS FOR CLOSED-LOOP NEUROMODULATION USING MULTIPLE BIOLOGICAL SIGNALS

(71) Applicant: The Feinstein Institutes for Medical Research, Manhasset, NY (US)

(72) Inventor: Timir B. DATTA-CHAUDHURI, Manhasset, NY (US)

(21) Appl. No.: 19/130,899

(22) PCT Filed: Nov. 17, 2023
(86) PCT No.: PCT/US2023/080266

§ 371 Date: May 16, 2025

Related U.S. Application Data
(60) Provisional application No. 63/426,364, filed on Nov. 17, 2022.

(51) Int. Cl. A61N 1/378; A61N 1/36

(52) U.S. Cl. CPC A61N 1/3787; A61N 1/36139; A61N 1/36171

(57) ABSTRACT

Neuromodulation devices and methods configured to supply electrical stimulation to a subject based on the one or more biological signals obtained by one or more sensors. These apparatuses may be configured to transmit more than 90% of the time, with limited and prescribed receiving capability.

### CLAIM OF PRIORITY

This patent application claims priority to U.S. provisional Application no. 63/426,364, titled “SYSTEMS AND METHODS FOR CLOSED-LOOP NEUROMODULATION USING MULTIPLE BIOLOGICAL SIGNALS,” and filed on Nov. 17, 2022, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Neuromodulation often requires complex devices and methods of treatment, and this complexity may limit the ability to implement neuromodulation in a variety of therapeutic and research situations. Acquisition of biological activity and response to neuromodulation therapy can involve sensors and electrodes that are often prohibitive to use in small animal models and result in designing analogous devices in the animal model and translation to a clinically useful device for human patients. For example, a neuromodulation device must include a power subsystem, charging subsystem, communication subsystem, and sensing subsystem; traditionally these devices must apply energy to a subject. Thus, large batteries have traditionally been required to supply sufficient power and maintain sufficient charge for application of electrical stimulation and operation of the system, since batteries typically do not scale well, and smaller batteries have disproportionally less energy stored in them. In most neuromodulation systems the batteries often occupy the majority of space within the volume of the implant. In addition to use in animal models, these devices may be optimizing for safe and effective deployment in human patients in order to minimize adverse impact associated with large and cumbersome neuromodulation devices.

For these reasons, it would be desirable to provide improved devices, systems, and methods for neuromodulation therapy. It would be particularly desirable to provide optimized charging systems, control and communication systems capable of being housed within a package optimized for the particular anatomic deployment. At least some of these objectives will be met by the various embodiments that follow.

### SUMMARY OF THE DISCLOSURE

Generally, a neuromodulation device can have a power source that may be configured to be wirelessly charged (e.g., by inductive charging, magnetic coupling, and/or magnetic resonance coupling), one or more sensors configured to obtain one or more biological signals from a subject; a processing unit operably connected to the one or more sensors, wherein the processing unit can be electrically coupled to the power source; and one or more neurostimulators in communication with the processing unit, each of the one or more neurostimulators configured to supply electrical stimulation to anatomy of the subject based on the one or more biological signals obtained by the one or more sensors.

In some examples, the power source, processing unit, one or more neurostimulators, and one or more sensors can be contained within a housing less than 3 cm³ (e.g., 2.5 cm³ or less, 2.0 cm³ or less, 1.9 cm³ or less, 1.8 cm³ or less, 1.7 cm³ or less, 1.6 cm³ or less, 1.5 cm³ or less, 1.0 cm³ or less, etc.). In some examples, the neuromodulation device can be implanted into anatomy of the subject. In some examples, the neuromodulation device can be configured to communicate wirelessly with one or more remote devices configured to adjust one or more operation parameters of the device. In some examples, the neuromodulation device further comprises a plurality of stimulation engines, each of the plurality of stimulation engines operably coupled to one or more of the neurostimulators.

In some examples, the stimulation engines can be configured to coordinate stimulation via the one or more neurostimulators. In some examples, each of the one or more neurostimulators can be configured to supply electrical stimulation having distinct stimulation characteristics coordinated by one or more of the stimulation engines. In some examples, at least one of the one or more sensors can be selected from the group consisting of a temperature sensor, potentiometer, potentiostat, pressure sensor, inertial measuring unit or other biopotential sensor (e.g., electromyography sensor, electrocardiogramsor, neural sensor, etc.). As mentioned above, in some examples, the device can be contained within a housing of 3.0 cm³ or less (e.g., 2.5 cm³ or less, 2.0 cm³ or less, 1.9 cm³ or less, 1.8 cm³ or less, 1.7 cm³ or less, 1.6 cm³ or less, 1.5 cm³ or less, 1.0 cm³ or less, etc.).

In some examples, the device can be entirely contained within a 2.2 cm³ housing. In some examples, the stimulation supplied by the one or more neurotransmitters can be responsive to a biological signal sensed by the one or more sensors. In some examples, at least one of the one or more sensors can be configured to sense a tachycardie event, wherein the stimulation is configured to reduce a heart rate of the subject.

In some examples, at least one of the one or more sensors can be an inertial measurement unit, and wherein the stimulation can be configured to reduce involuntary subject movement associated with a disease state. In some examples, at least one of the one or more sensors can be electrochemical sensor, wherein the stimulation is supplied based on one or more biochemical signals within the subject. In some examples, at least one of the one or more sensors can be configured to sense one or more biomolecules, wherein the stimulation is supplied based on the presence of one or more predetermined biomolecules.

In some examples, at least one of the one or more sensors can be a pressure sensor, wherein the stimulation is configured to modulate pressure of one or more body regions, or portions of a body region, such as (but not limited to) one or more blood vessels, one or more gastrointestinal vessels, pressure within an organ (e.g., brain, etc.). In some examples, at least one of the one or more sensors can be configured to sense a tremor. In some examples, the stimulation can be configured to modulate blood glucose levels. In some examples, the one or more biological signals related to the sensed tremor can be stored within the neuromodulation device, wherein the stored biological signals can be configured to generate a profile, and wherein stimulation can be supplied based on the generated profile in advance of a subsequent tremor.

In some examples, the stimulation can be supplied based on an adaptive closed-loop regime. In some examples, the device can be configured to operably communicate with one or more nerves (e.g., nerve fibers) and/or one or more muscle fibers, wherein selective stimulation is configured to be supplied to one or more nerves and/or muscle fibers. In some examples, the one or more biological signals can be associated with a disease. In some examples, one or more of the sensors can be configured to obtain one or more neural signals, wherein the processing unit can be configured to interpret the one or more neural signals, and wherein stimulation can be supplied based on the one or more interpreted neural signals. In some examples, at least one of the neurostimulators can be configured to supply electrical stimulation to the vagus nerve. In some examples, the device further comprises a nerve cuff operably connected to the processing unit. In some examples, the one or more neurostimulators can be configured to selectively supply electrical stimulation to one or more nerves based on the tissue morphology.

In some examples, neuromodulation device can be configured to be at least partially implanted into a subject, the neuromodulation device comprising a housing less than 3 cm³, wherein a processing unit and power source can be contained entirely within the housing, the processing unit operably coupled to one or more neurostimulators configured to supply electrical stimulation based on one or more biological signals sensed by one or more sensors; a computing device configured to communicate with the neuromodulation device configured to receive data from the neuromodulation device based on the one or more biological signals. In some examples, the computing device can be configured to develop one or more stimulation regimes based upon received data. In some examples, the neuromodulation device further comprised a plurality of stimulation engines operably connected to the processing unit and at least one of the neurostimulators. In some examples, the neuromodulator can be configured to be charged wirelessly, e.g., by magnetic resonance coupling.

In any of these apparatuses (e.g., devices, systems, etc.), the communications sub-system may be configured to transmit more than receive. For example, the apparatus may be configured to transmit (e.g., data from the one or more sensors, etc.) so that 85% or more of the communication traffic is transmission from the apparatus (with 15% or less receiving), e.g., 87% or more, 88% or more, 89% or more, 90% or more, 92% or more, 95% or more, etc. In practice, the apparatus may be configured to transmit more than 90% of the time (e.g., more than 92% of the time, more than 95% of the time, more than 97% of the time, more than 98% of the time, more than 99% of the time, etc.). Thus, the communications sub-system may be skewed to transmitting, rather than receiving.

In some examples the apparatus may be configured so that the control, or the communications subsystem is configured so that the apparatus “listens” for received commands/data at prescribed times, and/or only after transmitting a signal indicating that it will be “listening” for a received transmission withing a predetermined window of time. This configuration may reduce the power requirements and may optimize the operation of the apparatus.

In general, a bidirectional neuromodulator device can comprise a housing less than 3 cm³; a power source configured to be wirelessly charged (e.g., by inductive coupling, magnetic coupling, magnetic resonance coupling, etc.); one or more sensors configured to obtain one or more biological signals from a subject; a processing unit operably connected to the one or more sensors, wherein the processing unit can be electrically coupled to the power source; one or more neurostimulators in communication with the processing unit, each of the one or more neurostimulators configured to supply electrical stimulation to anatomy of the subject based on the one or more biological signals obtained by the one or more sensors.

In some examples, the housing can be coated with parylene. In some examples, the exterior of the housing can be hydrophobic. In some examples, the neuromodulation device further comprises an amplifier operably connected to the processing unit, the amplifier configured to amplify electrical signals produced by biological processes or signals from any of one or more sensors connected to the system. In some examples, the neuromodulation device further comprises a plurality of stimulation engines configured to produce biphasic stimulation. In some examples, the one or more of the neurostimulators can be configured to supply stimulation at a frequency of at least 10 kHz. In some examples, the one or more neurostimulators can be configured to apply current selectively to adjacent biological tissue.

In general, a method of neuromodulation can comprise implanting a bidirectional neuromodulator having a plurality of stimulation engines configured to coordinate a supply of electrical stimulation based on one or more biological signals; acquiring data from one or more biological signals associated with a subject; coordinating a stimulation regime based on the one or more biological signals; supplying current-mode electrical stimulation with one or more neurostimulators operably coupled to one or more of the plurality of stimulation engines, wherein the electrical stimulation is supplied based on the coordinated stimulation regime and wherein the neuromodulator is substantially contained within a housing less than 3 cm³. As used herein, a stimulation engine may include all or a portion of a pulse generator and/or other source of neuromodulation that may be in communication with one or more electrodes for applying neuromodulation to the subject. A stimulation engine may include one or more circuits that are configured to drive neuromodulation from one or more (e.g., a pair, a subset, etc.) of electrodes. Multiple stimulation engines may be included to allow the application of electrical energy to different electrodes or subsets of electrodes having different properties (e.g., pulse width, pulse duration, frequency, etc.).

In some examples, the method further comprises recording data based on the one or more biological signals within the neuromodulator. In some examples, the one or more biological signals are voltage signals including one or more directly transduced neural signals, indirectly sensed neural signals, or myoelectrical signals. In some examples, the one or more biological signals include at least one or more biochemical signals. In some examples, the method further comprises sensing one or more biomolecules, wherein the neuromodulator can be configured to identify the one or more biomolecules by impedance spectroscopy.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is an example of a neuromodulation device, as described herein, illustrated with a transparent housing exposing internal components.

FIG. 2 illustrates an example of a neuromodulation device in a partially expanded view with housing elements separated from one another to expose the internal electronics.

FIG. 3 shows an example of a neuromodulation device without a housing showing the printed circuit boards, power source, and electronic elements that can be incorporated for sensing and neurostimulation, as described herein.

FIG. 4 illustrates an example of a loop or system interaction with one or more remote devices communicating with a neuromodulation device, as described herein.

FIGS. 5A and 5B illustrate examples of operational architecture of a neuromodulation device, as described herein.

FIG. 6 illustrates an expanded view of elements comprising an example of a neuromodulation devices, as described herein.

FIG. 7 shows an example of a housing for a neuromodulation device compared to an example of a neuromodulation device that may be contained within the housing, as described herein.

FIG. 8 illustrates an example of neuromodulation capabilities presented in a diagram highlighting structural elements related to a charging system and a neuromodulation device, as described herein.

FIG. 9 is a graphical representation of sensor system examples that can be employed by a neuromodulation device, as described herein.

FIG. 10 illustrates examples of neuromodulation device placement including multiple neuromodulation devices locatable within a subject.

FIG. 11 is a diagram illustrating an example of neuromodulation device operation in fiber selective stimulation according to examples described herein.

FIG. 12 is a diagram illustrating an example of neuromodulation device operation in blood glucose and/or anti-inflammatory stimulation according to examples described herein.

FIG. 13 is a diagram illustrating an example of neuromodulation device operation in hypotensive stimulation to lower blood pressure according to examples described herein.

FIG. 14 is a diagram illustrating an example of neuromodulation device operation in behavioral conditioning stimulation according to examples described herein.

### DETAILED DESCRIPTION

A neuromodulation device can have one or more interventional systems in operable communication with a controller and a power source (e.g., an energy storage device, such as a battery, capacitive storage, etc.). One or more circuit boards (e.g., printed circuit boards or PCBs) can receive, retain, and route electrical connections between the power source, controller and one or more interventional systems. One or more sensors in operable communication with the neuromodulation device can be configured to obtain or sense biological attributes (e.g., biometric data) for processing by a computing engine in communication with the controller. A housing can be configured to receive one or more components of a neuromodulation device.

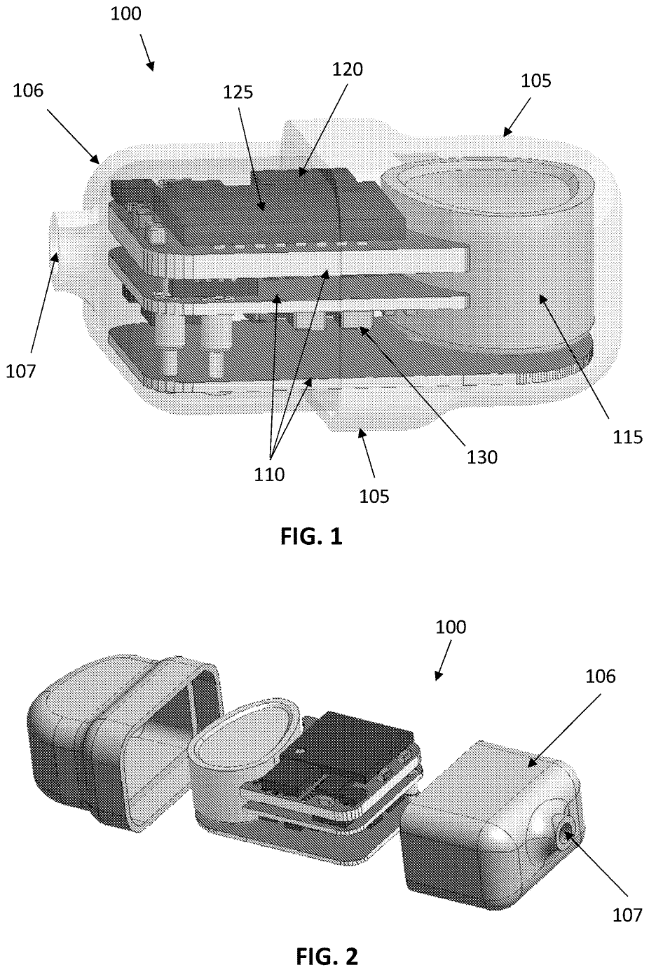

FIG. 1 illustrates an example of a neuromodulation device 100 described herein. A housing 105 can have one or more housing segments configured to engage one another to enclose or encapsulate one or more PCBs 110 and a power source 115. A first housing segment 106 is shown having a feedthrough (e.g., lumen) 107 extending from an interior of the housing to the exterior. The lumen 107 may be configured to facilitate the passage of one or more interventional systems for placement within a subject's anatomy. Here, three PCBs 110 are shown in an arrangement generally co-planar with one another to maximize organizational arrangements of the processor 120, controller 125, power source 115, one or more amplifiers 130, and power source charging system. A processor includes hardware that runs the computer program code. Specifically, the term ‘processor’ may include or be part of a controller and may encompass not only computers having different architectures such as single/multi-processor architectures and sequential (Von Neumann)/parallel architectures but also specialized circuits such as field-programmable gate arrays (FPGA), application specific circuits (ASIC), signal processing devices and other devices.

A neuromodulation device can have a power source charging system. In some examples, the charging system is configured to charge a battery coupled to a PCB of the neuromodulation device. An example of a power source charging system can be a wireless charging means (e.g., a magnetic resonance charging system). In some examples, the neuromodulation device can have a power receiving device configured to wirelessly receive power configured to charge the neuromodulation device power source. A power receiving device can be one or more wireless power receiving coils configured to receive power from one or more wireless power transmission devices having one or more transmission coils configured to remotely charge the neuromodulation device power source.

In some examples, the magnetic resonance charging system can be configured to wirelessly transfer power to the energy storage device (e.g., a rechargeable battery) of a neuromodulation device. In some examples, the quantity of power transmission coils of the magnetic resonance charging system may be greater than, less than, or equal to the quantity of power receiving coils in operable communication with the neuromodulation device power source. For example, a wireless charging system described herein can have four coils, where the wireless power transmitter and the wireless power receiver each comprise two coils. A wireless power transmitter can have a coil driven by a signal source that can be inductively coupled to one or more tunable resonant coils. The resonant coils can be tuned using one or more capacitors. In some examples, the wireless power receiver can have one or more tunable resonant coils that can be inductively coupled to a load coil. The load coil can be electrically connected to the neuromodulation device electronic circuitry (e.g., one or more PCBs). In some examples, a wireless charging system for a neuromodulation device can have an operation frequency between 0.01 and 10 MHz. For example, an operation frequency of a neuromodulation device operation frequency can be between 5 MHz and 8 MHz. In some examples, an operation frequency of a wireless charging system for a neuromodulation device can be 6.78 MHz.

In some examples, a wireless charging system for a neuromodulation device can have one or more amplifiers configured to modulate power signals. An example of an amplifier may be a differential amplifier with Gallium Nitride field effect transistor configured to generate high power signals to drive the source coil (e.g., power transmission coil).

In some examples, a wireless charging transmission device described herein can have a first coil in the transmission (e.g., source) side. The first coil can be single tuned and in communication with an amplifier. A second coil can be tuned to a resonant frequency can comprise thin electrically conductive material (e.g., copper foil). The wireless power receiving device can also have two coils. For example, two coils in a single planar flex PCB. The receiver coil can be tuned to resonant frequency. One of the receiver coils can be a load coil in electrical communication with the neuromodulation device circuitry and configured to transfer power to charge the neuromodulation device power source.

In some examples, a neuromodulation device and wireless charging system described herein may be deployed in an animal (e.g., a mouse). For example, a wireless charging system described herein can have one or more coils (e.g., two coils) on the transmission device that can be wrapped around a box configured to receive or house a mouse cage. A neuromodulation device associated with mice inside of the cage can receive wireless power transmitted from the one or more coils and received by the power receiving coils of the neuromodulation device.

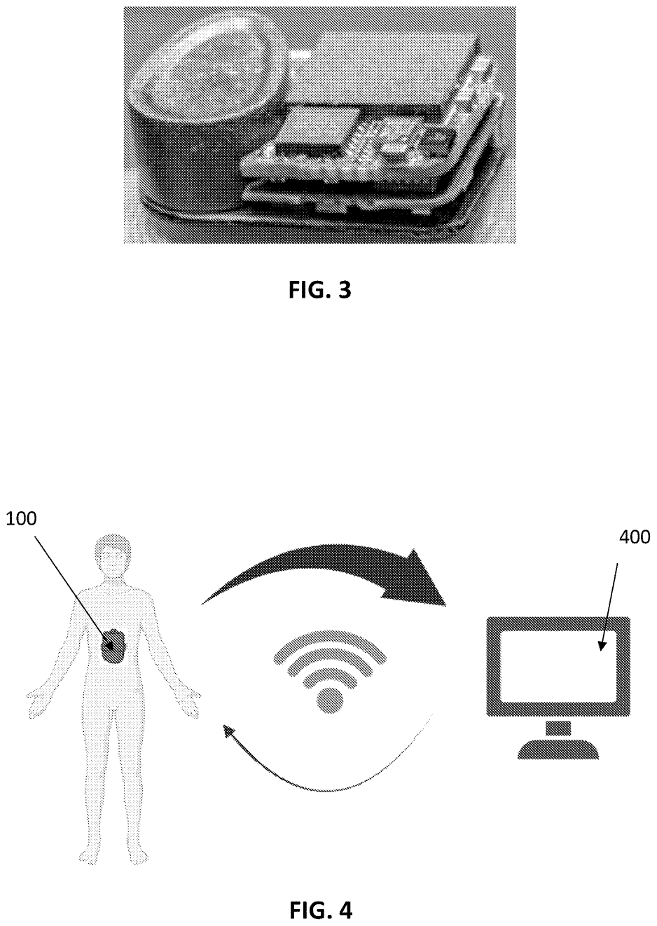

In FIG. 2, a neuromodulation device is illustrated with two halves of a housing 105 separated from one another exposing the electrical components of the neuromodulation device 100. Multiple PCBs 110 are shown with the processor 120, power source 115, and additional elements associated with the neuromodulation device function. The housing lumen 107 can be seen without an interventional element passing therethrough. Similarly, FIG. 3 shows the neuromodulation device without the housing.

A neuromodulation device described herein may have a data transmission system configured to communicate with one or more remote devices. In some examples, communication with one or more remote devices may be the transmission of data obtained by the neuromodulation device (e.g., data obtained by one or more sensors). In some examples, communication with one or more remote devices may be the transmission of operational instructions. For example, a remote device may be configured to generate a treatment regimen and transmit instructions for application of the treatment regime to the device.

In some examples, the transmission system is a wireless communication system configured to transmit data to and from the neuromodulation device in real-time. The wireless communication system can include packaging and transmission of packaged data at a rate sufficient for monitoring biometric data, response to application of neuromodulation device operations, and/or information for development of a treatment or operation regime.

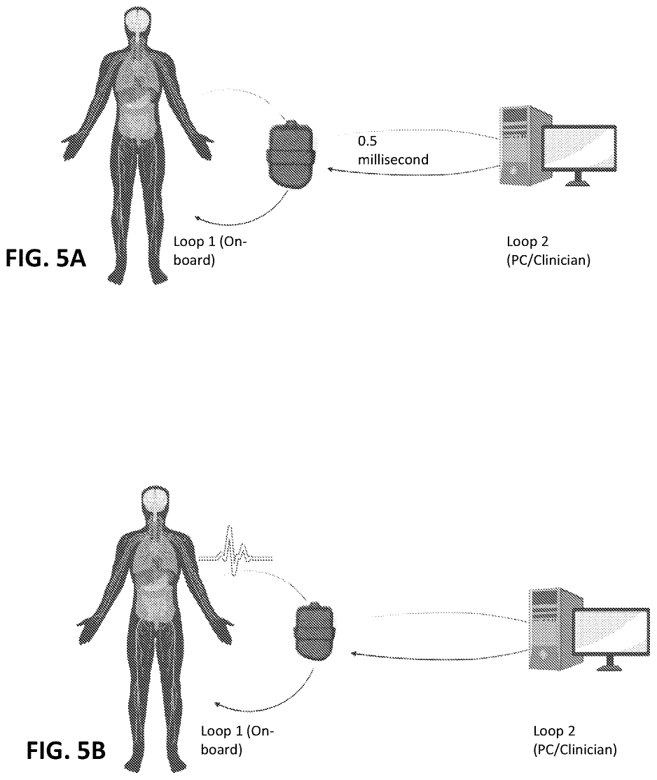

FIG. 4, FIGS. 5A and 5B illustrate examples of operational loops related to the operation and control of a neuromodulation device. FIG. 4 is an example of a neuromodulation device 100 operably located on or within a subject and wireless communicating with a remote device 400. Similarly, FIGS. 5A and 5B illustrate multiple loops of operation for a neuromodulation device within a patient. A first loop (e.g., a closed loop) may comprise operations of the neuromodulation device including the sensing of biometric data from the one or more sensors and processing of the sensed data by the processor within the neuromodulation device. In some examples, a second loop can be communication from a remote device operable by a healthcare professional to monitor, augment, instruct, or generate treatment regimes employed by the neuromodulation device. In some examples, the second loop may transmit data and instruction to the neuromodulation device within a subject that may augment or modify threshold values, stimulation characteristics (e.g., intensity, duration, etc.). In response to the augmented threshold values, the first loop may be adjusted.

In some examples, interventional systems of a neuromodulation device can be an electrical stimulation system having one or more electrodes configured to supply electrical stimulation to a subject. The electrical stimulation system may include a plurality of electrodes operably coupled to the controller and in communication with anatomy of the subject. may extend from within the housing to contact or communicate with anatomy of a subject. The electrodes can be in operable communication with a controller configured to direct application of an electrical stimulation according to a stimulation regime. In some examples, the electrodes may be configured to contact one or more nerves of a subject. For example, one or more electrodes may be a nerve cuff electrode. In some examples, one or more electrodes can be chronic ECG electrodes.

A stimulation regime may be predetermined and application of electrical stimulation by the one or more electrodes can be stored within the neuromodulation device. In some examples, a predetermined stimulation regime can be transmitted from one or more remote devices in communication with the neuromodulation device. The stimulation regime can include electrical stimulation attributes. For example, a stimulation regime may comprise intensity, duration, and/or stimulation distribution pattern.

In some examples, interventional systems of a neuromodulation device can be a biochemical system including one or more biochemical sensors configured to sense biometric data associated with biochemical molecules within the subject. For examples, a glucose monitoring sensor may be operably coupled to a neuromodulation device and configured to sense biometric data related to glucose levels in one or more biological tissues. Based on the biometric data from the one or more biochemical sensors, a neuromodulation device may supply electrical stimulation and/or may be triggered to release one or more therapeutic compounds to the subject from the neuromodulation device.

In some examples, interventional systems of a neuromodulation device can be a biopotential system with one or more sensors configured for electroencephalogram (EEG), electrocardiogram (ECG) and electromyogram (EMG) signal monitoring. Accordingly, the one or more biopotential sensors can be configured to trigger operation of the neuromodulation device to provide stimulation according to the sensed biopotential signals.

In some examples, interventional systems of a neuromodulation device can be a physical sensing system including one or more sensors configured to sense physical signals or mechanical changes in the subject. For example, inertia sensors may be operably connected to the neuromodulation device and configured to sense tremors occurring in the subject. In response to the sensed physical signal, a neuromodulation device can be signaled, and a responsive stimulation regime can be initiated to prevent, reduce, or react to a sensed physical biometric signal.

Components of a neuromodulation device described herein may be distributed on a plurality of PCBs arranged to organize elements of the neuromodulation device. For example, the charging system operably coupled to the power source can include one or more charging coils distributed on one or more layers of PCBs. In some examples, a first PCB layer can be operably coupled to the power source (e.g., battery) and configured to transfer power to one or more additional PCB layers. For example, a power source may be coupled to a first PCB and one or more electrodes may extend from a second PCB layer to a target location of a subject's anatomy and the battery can supply sufficient power for electrical stimulation via the one or more electrodes.

In some examples, the size of the housing can be minimized. In some examples, it may be advantageous for a neuromodulation device housing to be minimized for installation into a subject's anatomy. In some examples, a neuromodulation device may have at least three PCBs. In some examples, a neuromodulation device may comprise a single PCB layer, two PCB layers, three PCB layers, four PCB layers, five PCB layers, or more.

In some examples, the quantity of PCB layers may be based on optimizing the dimensions of the housing. In some examples, a single PCB layer may be sufficiently large to retain components of a neuromodulation device (e.g., battery, controller, processor, etc.). In some examples, one or more components of a neuromodulation device may be coupled to a first PCB layer and one or more components of the neuromodulation device may be coupled to one or more additional PCB layers. For example, the housing may have a width less than 12000 μm and a length less than 16000 μm. In some examples, the housing may have a width less than 20000 μm, less than 18000 μm, less than 16000 μm, less than 14000 μm, less than 12000 μm, less than 10000 μm, less than 8000 μm, less than 6000 μm, less than 4000 μm, less than 2000 μm or any length therebetween. In some examples, the housing may have a length less than 20000 μm, less than 18000 μm, less than 16000 μm, less than 14000 μm, less than 12000 μm, less than 10000 μm, less than 8000 μm, less than 6000 μm, less than 4000 μm, less than 2000 μm or any length therebetween.

In some examples, a housing for a neuromodulation device can be constructed using a method of epoxy encapsulation based on a mold (e.g., a 3D printed mold of the housing). For example, based on the desired size of the neuromodulation device, components can be organized on one or more flex PCBs and dimensions of a housing to encapsulate the neuromodulation device can be determined. A 3D printed master of the housing can be produced and a mold (e.g., silicon mold) based on the 3D printed housing can be generated. Electronics of the neuromodulation device can be suspended in a cavity of the mold and the mold can be filled with epoxy. In some examples, the epoxy can be degassed, and heat cured. This method can produce a biocompatible implant surface and efficient housing design.

In some examples, electronics of a neuromodulation device can be cleaned with alcohol (e.g., isopropyl alcohol) and/or deionized water prior to encapsulation. In some examples, after being cleaned, heat may be applied to the electronics (e.g., the electronics can be baked). The heat can be sufficient to remove moisture prior to packaging.

In some examples, a conformal coating (e.g., parylene C) layer can be added during the encapsulation process. Additional silicone may be added near the electrical feedthroughs (e.g., housing lumen) as a strain relief. In some examples, a mesh (e.g., polyester mesh) can be coupled (e.g., affixed) to the housing as an anchor (e.g., an anchor point for sutures) to provide mechanical stability to the neuromodulation device within the subject.

In some examples, interventional system components passing through the housing lumen (e.g., electrode wires) may be connected to one or more PCBs with PCB pins. For example, after the encapsulation process, PCB pins may be electrically connected to either electrode. In some examples, conductive epoxy (e.g., epoxy used during the encapsulation process may be used to electrically connect the pins and electrode(s). In some examples, laser welded joints can be used to form the electrical connections at the housing lumen between the electrodes and the PCB.

In some examples, the housing is constructed using a polymer-based enclosure packaging. For example, a housing can be 3D printed in two halves to encase the electronics. In some examples, all seams and/or openings are sealed with epoxy. Due to the electronics being under active development, there is a substantial benefit to maintaining accessibility to the circuit for troubleshooting. In the event of a device failure, whether during saline soak testing or implantation, it is possible to retrieve the electronics from the enclosure and much more easily diagnose any problems. This also simplifies and reduces packaging time by eliminating the encapsulation process which requires a significant amount of labor due to the multistep process of mold fabrication and two-staged epoxy casting (which includes centrifugal degassing of the epoxy resin).

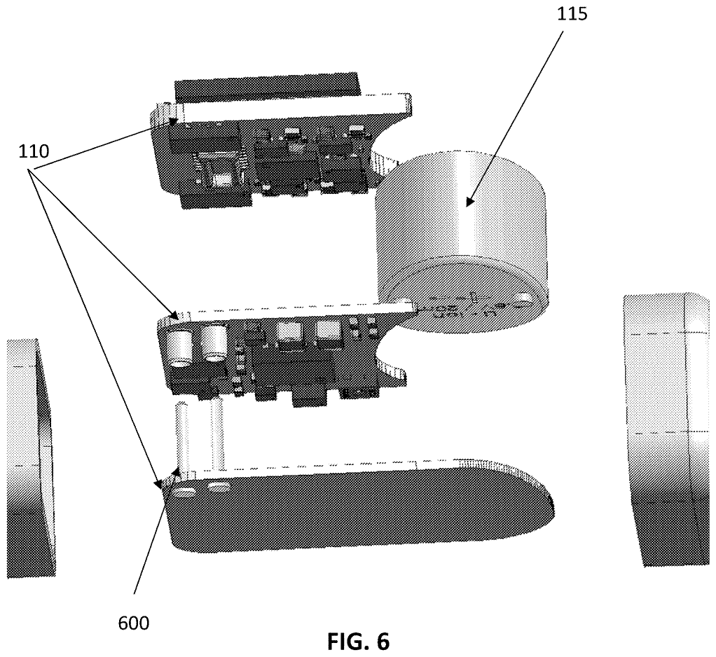

FIG. 6 illustrates an expanded view of a neuromodulation device 100. In this example the neuromodulation device includes a power source (e.g., lithium-ion rechargeable battery) 115. Pins 600 can be seen extending upward from the flex PCB to the additional PCBs.

An example of a neuromodulation device housing can be a multi-part (e.g., two-part), thin-walled, 3D printed frame made of biocompatible material. In some examples, a housing wall thickness can be 200 microns to 700 microns (e.g., 500 microns). After 3D printing, the separate halves of the 3D printed enclosure can be coated with conformal coating layer (e.g., parylene C). In some examples, the conformal layer can be hydrophobic. In some examples, the housing parts can be sealed using an adhesive, such as an epoxy (e.g., LOCTITE®).

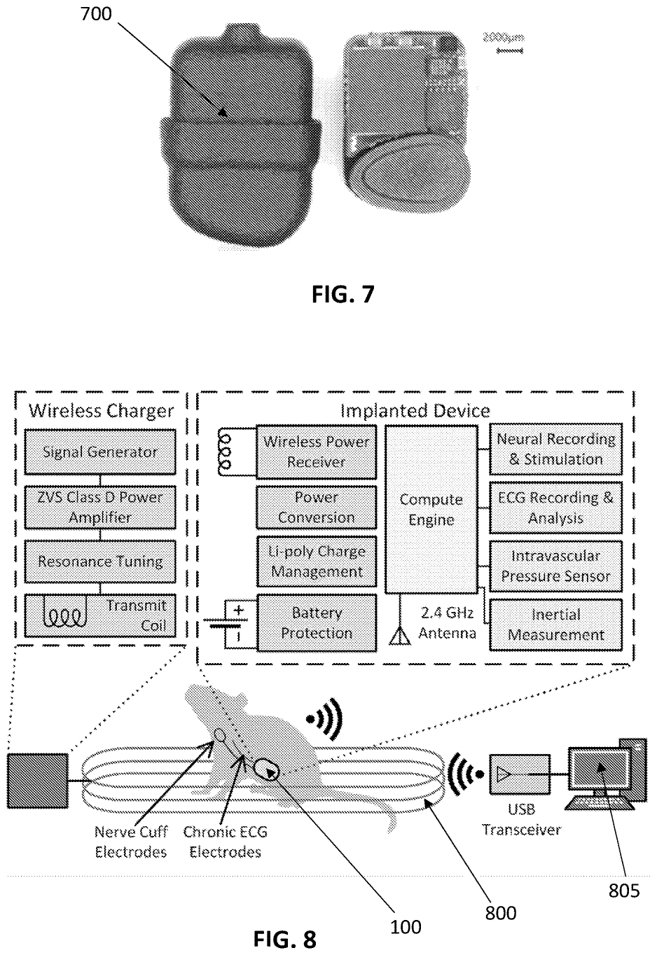

FIG. 7 is an image of a neuromodulation device outside of a housing 105. The housing has a first half and second half coupled to one another at a joint 700. The housing lumen 107 is shown at the terminal end of one of the housing halves. The neuromodulation device electrical components are removed from the housing to illustrate the size comparison between the electrical components and the housing design. As discussed herein, the housing design and characteristics can be associated with the dimensions of the neuromodulation device electrical components through one or more methods described herein.

In some examples, parts of the housing may engage one another at a joint. For example, a joint between housing parts may be a butt joint or lap joint.

The apparatus may be packaged in an inert configuration. For example, in some examples, the neuromodulation device electronics can be wrapped with polyimide tape (total thickness of 38 microns) prior to packaging. The polyimide tape can be configured to to prevent contamination of the electronic components.

In some examples, the neuromodulation devices described herein may be implanted within a subject such that the housing and internal systems are located at a site within a subject's anatomy. Implantation and placement of a neuromodulation device may be related to the target anatomical structures for application of interventional impact of the neuromodulation device. For example, intervention for peripheral neuromuscular stimulation can include implantation of a neuromodulation device at or near a peripheral nerve of a subject. FIG. 8 illustrates a schematic overview of examples of structures associated with a neuromodulation device. Here, a mouse is depicted and the neuromodulation device 100 is represented as being implanted in the mouse. A magnetic resonance wireless charging system 800, as described herein, is illustratively shown around the mouse and positioned to wirelessly transmit power to the receiving device of the neuromodulation device 100. A remote device 805 (e.g., computer) is illustrated to show wireless communication or transfer of data to and/or from the neuromodulation device.

Examples of components for a wireless charger are graphically represented as well as examples of components of the neuromodulation device including the wireless charging system, and computing engine in communication with the interventional systems and sensors.

An illustrative example of a neuromodulation device configuration may be a processing unit (e.g., Cortex M4) and a plurality of physical channels (e.g., four channels). The neuromodulation device may further comprise one or more stimulation engines (e.g., 16 stimulation engines) having a stimulation capability at greater than, less than, and/or equal to 10 kHz. The neuromodulation device may have an inertia sensor (e.g., IMU sensor), temperature sensor, humidity sensor, a potentiometer, and a physical sensor.

An example of a neuromodulation device PCB arrangement can be two component boards (a first PCB and a second PCB) connected by pin connectors (e.g., 16 pin molex mezzanine connectors) having signal and power lines. An additional flex PCB coil can be connected to the second PCB using pin connectors. Such a configuration can provide improved disassembly and augmentation of the neuromodulation device. In some examples, laser cut ferrite sheets can be used to block eddy current build up in other metal components that would decrease the efficiency of the power transfer.

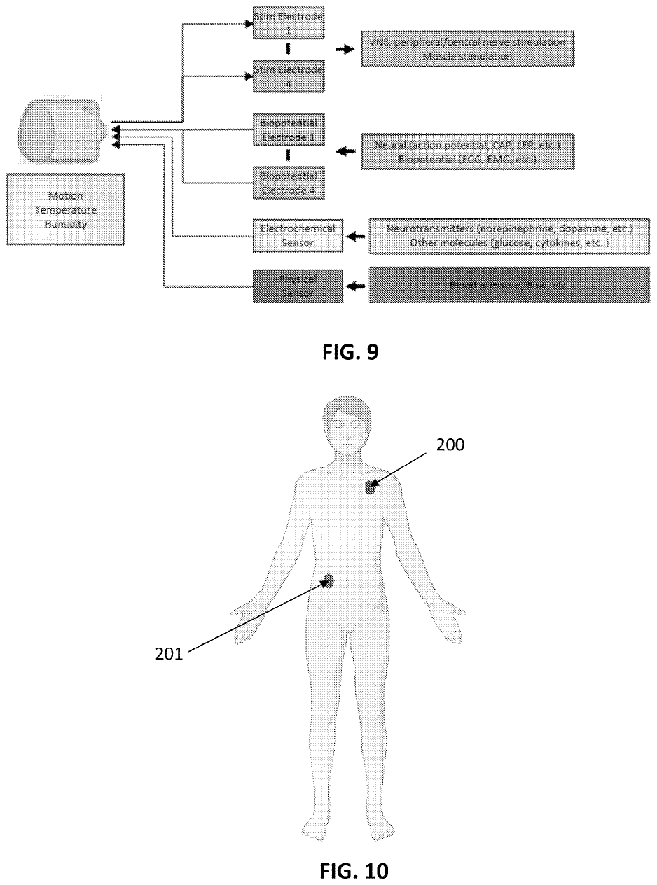

FIG. 9 illustrates examples of functional modules (e.g., interventional systems) of a neuromodulation device. The sensors and/or interventional systems described herein may be physically coupled to the neuromodulation device. In some examples, one or more of the interventional systems and/or sensors may be in wireless communication with a neuromodulation device. For example, one or more sensors and/or interventional systems may be locatable at a position or location of a subject's anatomy separate from the neuromodulation device housing. In some examples, sensors may be in remote communication with the neuromodulation device such that sensing of one or more remote anatomical locations may beneficially trigger operation and therapeutic application of electrical stimulation or other function of the neuromodulation device at a separate anatomical location (e.g., the implanted location of the neuromodulation device).

In some examples, and as illustrated in FIG. 10, multiple neuromodulation devices may be located or positioned at different anatomical locations of a subject. For example, more than one neuromodulation device 200, as described herein, may be positioned in the torso of a subject, with a separate neuromodulation device 201 positioned in the abdomen of the subject. Accordingly, each neuromodulation device may function independently of one another. In some examples, each neuromodulation device may communicate with one another to provide feedback or transmit data for operation of each neuromodulation device based on information acquired and aggregated from each implanted or deployed neuromodulation devices. For example, electrical signals sensed by a neuromodulation device within a subject's torso may transmit such data to a neuromodulation device positioned near a trunk of a nerve separate from the torso neuromodulation device.

### Example: Fiber Selective Stimulation

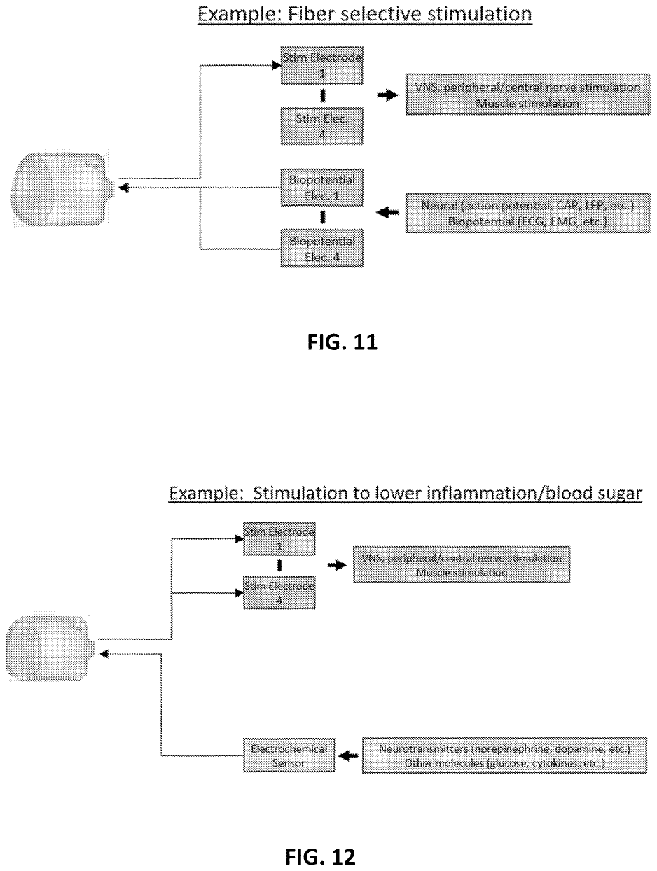

FIG. 11 illustrates an example of a neuromodulation device operation for fiber selective operation. A neuromodulation device described herein can be configured to selectively stimulate target biological tissue (e.g., selective fiber stimulation). The diagram illustrated in FIG. 11 shows a process including a neuromodulation device receiving input from one or more sensors. The sensors may be biopotential electrical sensors in operable communication with a subject's anatomy. One or more of the sensors can obtain neural biometric data (e.g., action potentials, combined action potential, local field potential, etc.). One or more sensors can obtain electromyographic (EMG) biometric data, electrocardiogramd/or additional biopotential data associated with a subject's biological tissues or systems. The sensed data is transmitted from the sensor to the neuromodulation device (e.g., the processor). The data can be interpreted by the neuromodulation device (e.g., the processor) and a stimulation plan can be established based on the sensed data. The neuromodulation device (e.g., the controller) can then direct electrical stimulation via one or more stimulation electrodes to target tissue.

### Example: Stimulation to Lower Inflammation and/or Blood Sugar

FIG. 12 illustrates an example of a neuromodulation device operation for stimulation to modulate inflammation and/or blood sugar. A neuromodulation device described herein can be configured to selectively stimulate target biological tissue (e.g., peripheral nerve and/or neuromuscular stimulation). The diagram illustrated in FIG. 12 shows a process including a neuromodulation device receiving input from one or more sensors. The sensors may be biochemical sensors in operable communication with a subject's anatomy. One or more of the sensors can obtain biochemical biometric data (e.g., inflammatory cytokine concentration, blood glucose concentration, norepinephrine, dopamine). The sensed data is transmitted from the sensor to the neuromodulation device (e.g., the processor). The data can be interpreted by the neuromodulation device (e.g., the processor) and a stimulation plan can be established based on the sensed data. The neuromodulation device (e.g., the controller) can then direct electrical stimulation via one or more stimulation electrodes to target tissue.

### Example: Stimulation to Lower Blood Pressure

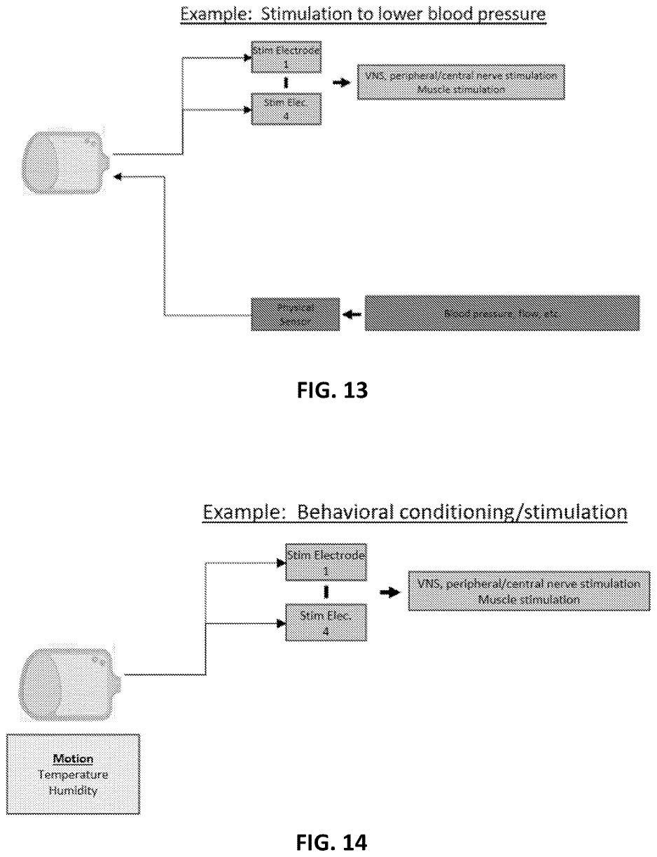

FIG. 13 illustrates an example of a neuromodulation device operation for stimulation to modulate blood pressure. A neuromodulation device described herein can be configured to selectively stimulate target biological tissue (e.g., peripheral nerve and/or neuromuscular stimulation). The diagram illustrated in FIG. 13 shows a process including a neuromodulation device receiving input from one or more sensors. The sensors may be physical sensors in operable communication with a subject's anatomy. One or more of the sensors can obtain physical biometric data (e.g., blood pressure, tremors, blood flow rate). The sensed data is transmitted from the sensor to the neuromodulation device (e.g., the processor). The data can be interpreted by the neuromodulation device (e.g., the processor) and a stimulation plan can be established based on the sensed data. The neuromodulation device (e.g., the controller) can then direct electrical stimulation via one or more stimulation electrodes to target tissue.

### Example: Behavioral Conditioning and/or Stimulation

FIG. 14 illustrates an example of a neuromodulation device operation for behavioral conditioning. A neuromodulation device described herein can be configured to selectively stimulate target biological tissue (e.g., selective fiber stimulation). The diagram illustrated in FIG. 14 shows a process including a neuromodulation device configured to operate separate from one or more sensors. For example, electrical stimulation may be selectively applied or applied on-command without need for biometric data from one or more sensors. Alternatively or additionally, in some examples one or more sensors may be used to trigger operation (e.g., neurostimulation) by the device. The sensors may be operable to acquire data in response to the manual application of electrical stimulation. For examples, the neuromodulation device may be controllable by a subject for application of stimulation or other interventional system as desired. As illustrated in FIG. 14, application of electrical stimulation to the subject (e.g., the subject's nervous and/or neuromuscular system) may be to induce a response or deter activity of the subject associated with the neuromodulation device.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control/perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device.

Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium”, as used herein, generally refers to any form of device, carrier, or medium capable of storing or carrying computer-readable instructions.

Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising”, when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately”, even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-45. (canceled)

46. A bi-directional neuromodulator device comprising:
a housing less than 3 cm³;
a power source configured to be wirelessly charged;
one or more sensors configured to obtain one or more biological signals from a subject;
a processing unit operably connected to the one or more sensors, wherein the processing unit is electrically coupled to the power source; and
one or more neurostimulators in communication with the processing unit, each of the one or more neurostimulators configured to supply electrical stimulation to anatomy of the subject based on the one or more biological signals obtained by the one or more sensors.

47. The device of claim 46, wherein the housing is coated with a conformal coating.

48. The device of claim 46, wherein the exterior of the housing is hydrophobic.

49. The device of claim 46, further comprising an amplifier operably connected to the processing unit and the one or more neurostimulators, the amplifier configured to amplify electrical stimulation supplied by the one or more neurostimulators.

50. The device of claim 46, further comprising a plurality of stimulation engines configured to produce biphasic stimulation.

51. The device of claim 46, wherein one or more of the neurostimulators is configured to supply stimulation at a frequency of at least 10 kHz.

52. The device of claim 46, wherein the one or more neurostimulators are configured to selectively direct current to adjacent biological tissue.

53. A method of neuromodulation, the method comprising:
implanting a bidirectional neuromodulator having a plurality of stimulation engines configured to coordinate a supply of electrical stimulation based on one or more biological signals;
acquiring data from one or more biological signals associated with a subject;
coordinating a stimulation regime based on the one or more biological signals; and
supplying current-mode electrical stimulation with one or more neurostimulators operably coupled to one or more of the plurality of stimulation engines, wherein the electrical stimulation is supplied based on the coordinated stimulation regime and wherein the neuromodulator is substantially contained within a housing less than 3 cm³.

54. The method of claim 53, further comprising sensing interference-based response of one or more biological tissues receiving the supplied electrical stimulation, wherein the neuromodulator comprising a plurality of independent stimulation channels.

55. The method of claim 53, further comprising amplifying the electrical stimulation, wherein the neuromodulator comprising an amplifier operably connected between a processing unit and the one or more neurostimulators.

56. The method of claim 53, further comprising recording data based on the one or more biological signals within the neuromodulator.

57. The method of claim 53, wherein the one or more biological signals are voltage signals including one or more directly transduced neural signals, indirectly sensed neural signals, or myoelectrical signals.

58. The method of claim 53, wherein the one or more biological signals include at least one or more biochemical signals.

59. The method of claim 53, further comprising sensing one or more biomolecules, wherein the neuromodulator is configured to identify the one or more biomolecules by impedance spectroscopy.

60. A neuromodulation system comprising:
a neuromodulation device configured to be at least partially implanted into a subject, the neuromodulation device comprising a housing less than 3 cm³, wherein a processing unit and power source are contained entirely within the housing, the processing unit operably coupled to one or more neurostimulators configured to supply electrical stimulation based on one or more biological signals sensed by one or more sensors; and
a computing device configured to communicate with the neuromodulation device configured to receive data from the neuromodulation device based on the one or more biological signals.

61. The system of claim 60, wherein the computing device is configured to develop one or more stimulation regimes based on received data.

62. The system of claim 60, wherein the neuromodulation device further comprised a plurality of stimulation engines operably connected to the processing unit and at least one of the neurostimulators.

63. The system of claim 60, wherein the neuromodulator is configured to be wirelessly charged.

64. The system of claim 60, wherein the system includes one or more neurostimulators, wherein each of the one or more neurostimulators are configured to supply electrical stimulation having distinct stimulation characteristics coordinated by one or more of the stimulation engines.

65. The system of claim 60, wherein at least one of the one or more sensors is selected from the group consisting of a temperature sensor, potentiometer, pressure sensor, inertial measuring unit or electrocardiogramaensor.
